(19) United States
(12) Patent Application Publication
(10) Pub. No.: US 2026/0199567 A1
(43) Pub. Date: Jul. 16, 2026

(54) BREAST SHIELD WITH FLEXIBLE PATHWAY FOR ENHANCED MILK EXPRESSION

(71) Applicant: Zev Supplies Corp., Pompano Beach, FL (US)

(72) Inventors: Dina Rendler, Lauderhill, FL (US); Jianbiao Chen, Shantou City (CN); Jishun Huang, Shantou City (CN)

(21) Appl. No.: 19/238,273

(22) Filed: Jun. 13, 2025

Related U.S. Application Data
(63) Continuation-in-part of application No. 19/238,255, filed on Jun. 13, 2025.

(30) Foreign Application Priority Data
Jun. 13, 2024 (CN) 2024213436906

(51) Int. Cl. A61M 1/06

(52) U.S. Cl. CPC A61M 1/064; A61M 1/069; A61M 2202/0014; A61M 2202/04; A61M 2205/3331; A61M 2210/1007

(57) ABSTRACT

A breast pump flange assembly with enhanced suction capability for more efficient milk expression. The assembly includes a structural skeleton formed of a base ring, first and second posts, and a rigid flange ring, providing rigidity and support. The flange includes a flexible tunnel extending from a flexible inner ring, which during operation, flexible tunnel walls move inward due to pressure differential, creating additional suction specifically at the nipple and drawing it further into the tunnel space. This dynamic response to pressure changes enhances milk expression while maintaining user comfort. Expressed milk flows through the flexible tunnel and base ring into an attached baby bottle for collection.

### RELATED APPLICATIONS

This application is a continuation-in-part of pending U.S. application Ser. No. 19/238,255, filed Jun. 13, 2025, which claims priority to pending Chinese Application No. 2024213436906, filed Jun. 13, 2024, the contents of which are incorporated by reference herein.

### FIELD OF THE INVENTION

The invention relates to breast pumps flanges, more specifically to a breast pump flange with improved suction and milk expression.

### BACKGROUND OF THE INVENTION

Breast pumps have become essential devices for nursing mothers, allowing for the collection and storage of breast milk when direct breastfeeding is not possible or practical. The efficiency and comfort of breast pumps are primarily determined by the flange component, which directly interfaces with the breast tissue and channels the expressed milk into a collection container.

Traditional breast pump flanges typically feature a rigid, conical shape with a circular opening that accommodates the nipple. These conventional designs rely primarily on the suction created by the pump mechanism to express milk from the breast. However, such designs often fail to efficiently mimic the natural sucking motion of an infant, which involves both suction and compression of the tissue.

Early breast pump designs utilized rigid flanges made from hard plastic materials that provided little flexibility or adaptability to different breast shapes and sizes. These one-size-fits-all approaches frequently resulted in discomfort, inadequate milk expression, and in some cases, tissue trauma or nipple damage due to improper fit or excessive suction.

More recent developments have included flanges with softer materials or inserts at the breast contact points. However, these improvements have largely focused on comfort rather than addressing the fundamental suction mechanics that drive efficient milk expression. Additionally, many existing designs fail to create the dynamic pressure changes that more closely simulate an infant's natural feeding patterns.

While various flange designs exist in the market, there remains a need for a flange system that combines structural support with flexible components that work in concert with the pump's suction mechanism to enhance milk expression efficiency. Existing flanges typically do not feature integrated components that actively respond to pressure changes during the pumping cycle to create additional targeted suction at the nipple.

### SUMMARY OF THE INVENTION

The present invention provides an improved breast pump flange designed to enhance milk expression efficiency through an innovative combination of rigid structural components and strategically positioned flexible elements. The improved flange creates a more effective suction mechanism that better mimics the natural feeding action of an infant.

In one aspect, the invention comprises a structural skeleton formed of a base ring, first and second posts extending from the base ring, and a rigid flange ring, all constructed of hard plastic material. This structural skeleton provides the necessary rigidity and support for the flexible components of the assembly.

In another aspect, the invention includes a flexible outer ring extending from the outer perimeter of the flange ring, and a flexible inner ring extending from an internal circumference of the flange ring. These flexible components provide a comfortable interface with breast tissue while maintaining an effective seal.

In yet another aspect, the invention comprises a flexible tunnel extending from the flexible inner ring. The tunnel has a wider portion at its attachment to the flexible inner ring and tapers to a substantially cylindrical section that terminates at the base ring. In embodiments of the invention, the flexible tunnel is configured to dynamically respond to pressure changes during the pumping cycle. Unlike prior breast shields that focus solely on extraction through a pull-and release motion the embodiments of the invention provide a flexible tunnel that mimics the mechanics of how a baby naturally breastfeeds. In this regard, research shows that when a baby nurses, the tongue creates a wave-like motion while generating suction, helping to stimulate and maintain milk flow. A tunnel portion of the a flange in accordance with embodiments of the invention is designed to replicate that motion, providing both gentle suction and tongue-like compression for enhanced milk expression.

Embodiments of the invention further include a pump connector that functions as an adaptor, connecting via a plastic tube to a pump unit. The pumping action creates a pressure differential that causes the flexible tunnel walls to move inward, generating additional targeted suction on the nipple and drawing it further into the space defined by the flange.

In operation, when the flange is applied to a breast with the nipple positioned in the center, and suction is applied by the pump unit, the flexible tunnel walls respond to the pressure differential by moving inward. This movement creates enhanced suction specifically at the nipple, causing it to be drawn further into the tunnel space, thereby increasing milk expression from the breast. The pump unit cycles between suction and no suction thereby recreating the sucking pattern of an infant.

The expressed milk flows through the flexible tunnel and base ring and is collected in an attached baby bottle for convenient storage.

The invention thus provides a significant improvement over conventional breast pump flanges by incorporating flexible components that dynamically respond to pressure changes, thereby enhancing suction efficiency while maintaining comfort for the user.

### BRIEF DESCRIPTION OF THE FIGURES

FIG. 1 is a top view of a breast pump flange in accordance with an embodiment of the invention.

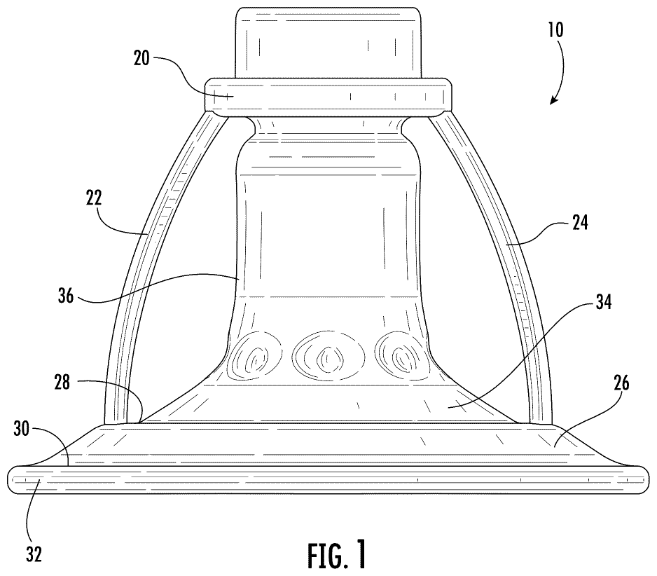

FIG. 2 is a side perspective view of a breast pump flange and bottle system in accordance with an embodiment of the invention.

FIG. 3 is a front perspective view of a breast pump flange in accordance with an embodiment of the invention.

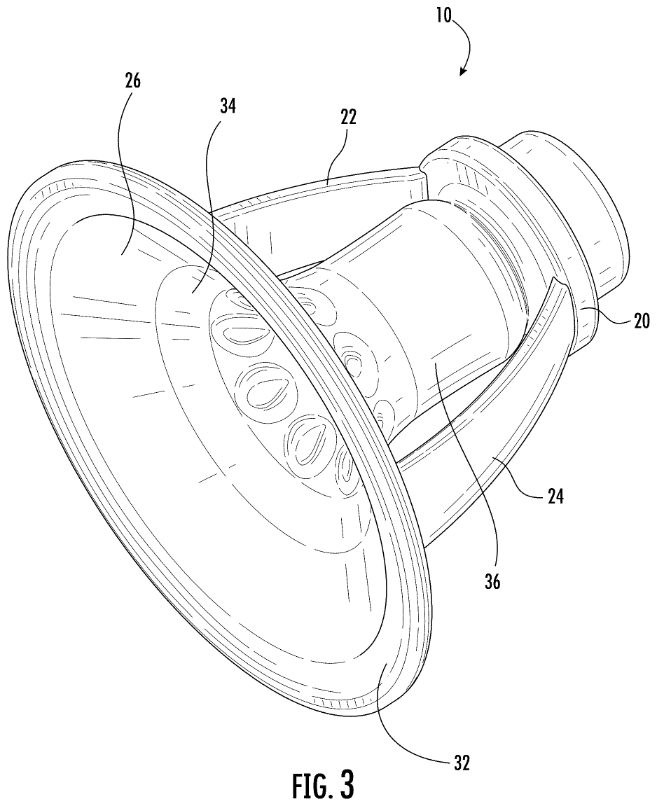

FIG. 4 is a top view of a breast pump flange shown applied to a breast in accordance with an embodiment of the invention.

FIG. 5 is a top view of a breast pump flange shown applied to a breast in accordance with an embodiment of the invention.

FIG. 6 a top view of a breast pump flange in accordance with an embodiment of the invention.

### DETAILED DESCRIPTION OF THE INVENTION

Embodiments of the present invention will now be described with reference to the above-identified figures of the drawings. However, the drawings and the description herein of the invention are not intended to limit the scope of the invention. It will be understood that various modifications of the present description of the invention are possible without departing from the spirit of the invention. Also, features described herein may be omitted, additional features may be included, and/or features described herein may be combined in a manner different from the specific combinations recited herein, all without departing from the spirit of the invention.

In embodiments of the invention, breast pump flange assembly 10 is formed of a structural skeleton that provides support and rigidity. In embodiments, the structural skeleton includes a base ring 20 and one or more support members. In the embodiment shown, a first post 22 and a second post 24 extending from the base ring 20. As shown, each of posts 22, 24 has a first end that is attached to base ring 20 and a second end that is connected to a rigid flange ring 26. The base ring 20, first post 22, second post 24, and flange ring 26 are formed of rigid plastic materials and provide structural rigidity and support for the flexible components of the assembly 10. In various embodiments, the structural skeleton may comprise a single post, two posts as shown, three or more posts, or alternative support configurations such as ribs, webs, or the like.

Alternative embodiments may include various ring configurations that provide equivalent sealing, dynamic, and interface functions. In some embodiments, the flange assembly may comprise a single flexible ring that extends from either the inner circumference 28 or the outer circumference 30 of the rigid flange ring 26, while maintaining effective sealing with breast tissue.

It will be understood that structural components may be constructed from hard plastic materials including, but not limited to, polypropylene, acrylonitrile butadiene styrene (ABS), polycarbonate, polyethylene or the like. In preferred embodiments, these materials are BPA-free and medical grade to ensure safety for both mother and baby.

As shown, in embodiments of the invention, rigid flange ring 26 has an inner circumference 28 and an outer circumference 30. In embodiments of the invention, a flexible outer ring 32, extends from the outer circumference 30 of the rigid flange ring 26, and a flexible inner ring 34 extends from the inner circumference 28 of the rigid flange ring 26. It will be understood that flexible inner ring 34 and the flexible outer ring 32 may be formed integrally with rigid flange ring 26. The flexible outer ring 32 which extends outwardly from the outer perimeter of the rigid flange ring 26 and is designed to comfortably contact the breast tissue.

In embodiments of the invention, the flexible portions of the flange assembly (e.g. outer ring 32, flexible inner ring 34, tunnel walls) may be constructed from any suitable flexible, biocompatible material including, but not limited to, silicone, thermoplastic elastomers (TPE), natural rubber, synthetic rubbers, flexible plastics, elastomeric compounds, or multi-material composites that provide appropriate flexibility and biocompatibility that provides a comfortable interface with breast tissue while maintaining sufficient durability for repeated use.

It will be understood that the portion of flange assembly that form a substantaiily concave or frusto conical “trumpet shaped” interior lumen may be referred to as a “flange interface,” whereas the entire unit may be referred to as a flange assembly.

A flexible tunnel 36 extends from the flexible inner ring 34 toward the base ring 20. In embodiments, the flexible tunnel 36 has a wider diameter at its point of attachment to the flexible inner ring 34 and tapers to a substantially cylindrical section that terminates at the base ring 20. Alternative embodiments may include a tunnel with uniform diameter, stepped diameter changes, oval cross-sections, or other geometric configurations that provide the desired contractile response to pressure differentials. The flexible tunnel 36 may also be constructed from silicone, TPE, or similar flexible materials that allow for appropriate expansion and contraction during the pumping cycle.

Flexible tunnel 36 defines a lumen that is in fluid communication with the concave area of the flange interface. Accordingly, when negative air pressure is generated in the tunnel (via pumping action of the pump), this negative pressure is transmitted through the lumen to the concave interior of the flange interface. (It should be noted that the terms “negative pressure,” or “reduced pressure,” refers to pressure levels below ambient atmospheric pressure at standard conditions.)

In embodiments of the invention, walls of tunnel 36 are flexible such that they are responsive to pressure differentials. In this regard, tunnel walls are configured to change in configuration (such as partially collapsing, compressing, contracting, or otherwise reducing cross-sectional area) as a result of negative or reduced pressure. The degree and nature of the response may vary depending on wall thickness, material properties, internal support structures, or other design parameters. Accordingly, when the flange is placed on a breast in a tight engagement, negative pressure will cause walls of tunnel to contract (i.e. tunnel wall is drawn toward a longitudinal axis of tunnel 36).

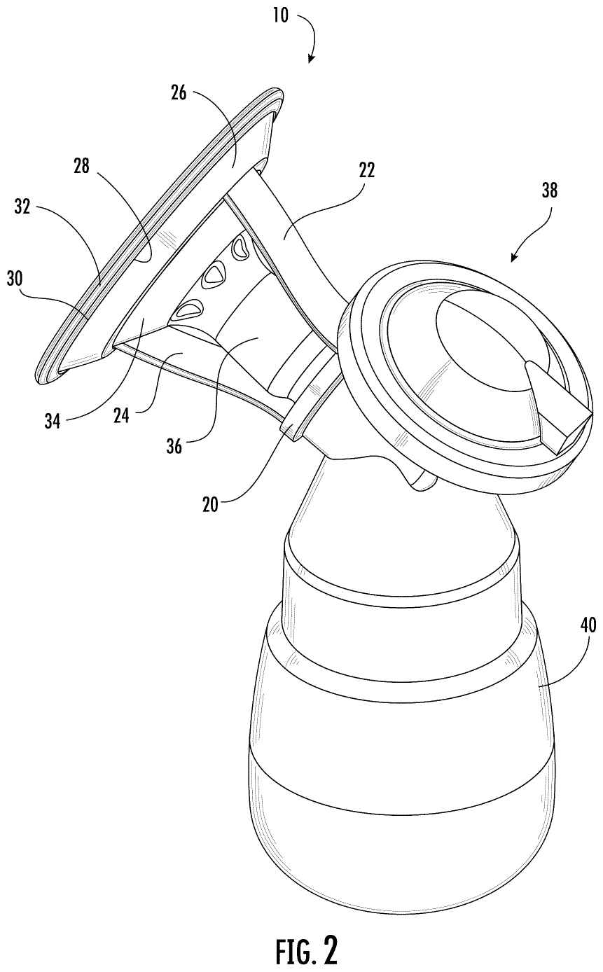

With reference to FIG. 2, a three-way pump connector 38 is integrated into the assembly, serving as an adaptor that connects the flange assembly 10 via a plastic tube to a pump unit (not shown). The pump connector 38 may be constructed from the same rigid plastic materials as the structural components or may incorporate both rigid and flexible elements to ensure a secure, airtight connection to external pumping equipment.

The breast pump flange 10 is designed to be used in conjunction with a baby bottle 40 for collecting expressed milk. The base ring 20 is configured to securely connect to the three-way pump connector 38 to attach a standard baby bottle through compatible threading or other connection mechanisms.

In operation, the flange interface of flange assembly 10 with its concave side is applied to a breast such that the nipple is positioned substantially in the center of the opening defined by the flexible inner ring 34. When suction is applied by the pump unit through the pump connector 38, a pressure differential is created within the assembly. The pressure differential creates a vacuum that draws milk from the breast. However, in addition, in embodiments of the invention, the pressure differential causes the flexible tunnel walls of tunnel 36 to contract (e.g. partially collapse), generating additional suction on the nipple. This enhanced suction gently draws the nipple farther into the space defined by the flange assembly 10. In embodiments of the invention enhanced suction provided by the contraction of the tunnel walls draws the nipple farther into the tunnel 36 with the application of negative pressure.

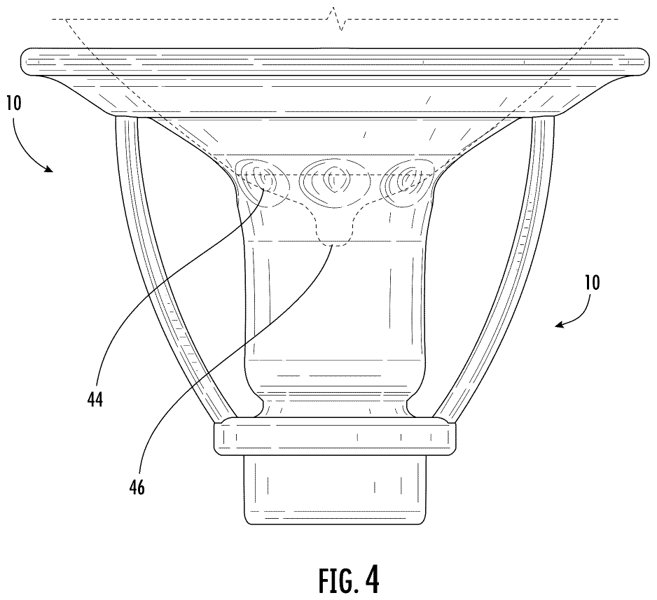

FIG. 4 shows a flange applied on a breast 44 during a time when there is no vacuum (i.e. the pump unit is quiescent). As shown, a segment of breast tissue is inserted within a concave aspect of the flange interface. It will be understood that nipple 46 may be positioned within the concave area of the flange interface or it may be positioned in the lumen of tunnel 36 (e.g. as shown).

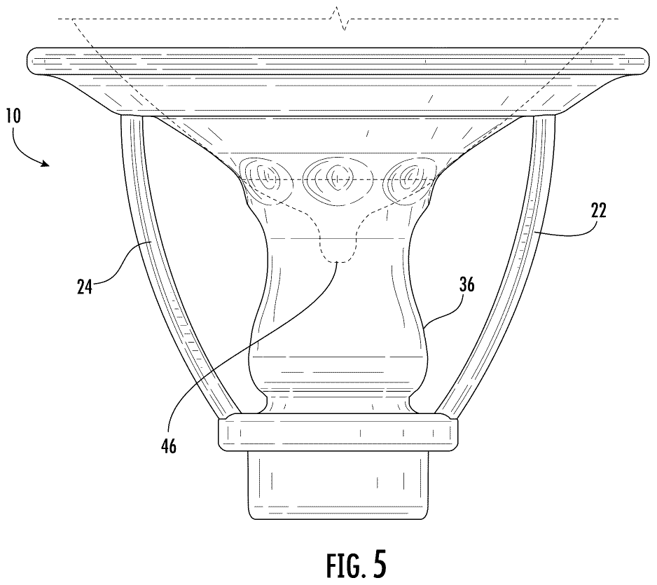

As shown in FIG. 5, when the pump cycles on to active pumping, negative pressure is generated in the tunnel 36 and flange interface. As shown, during active pumping, negative pressure draws nipple 46 and breast tissue farther into the lumen defined by tunnel 36 while simultaneously contracting the tunnel walls inward toward the center of the lumen. When the pump action terminates, the breast tissue returns to its starting position. This cycle repeats throughout the expression process.

The dynamic action of the flexible tunnel 36 during the pumping cycle mimics the natural suckling motion of an infant, thereby increasing milk expression from the breast. Milk expressed from the breast flows through the flexible tunnel 36 and base ring 20 and is collected in the attached baby bottle 40.

The improved design of the flange assembly 10 allows for more efficient milk expression while maintaining comfort for the user. The combination of rigid structural components and flexible contact surfaces provides both the necessary support and the gentle interaction with sensitive breast tissue that is essential for effective and comfortable pumping.

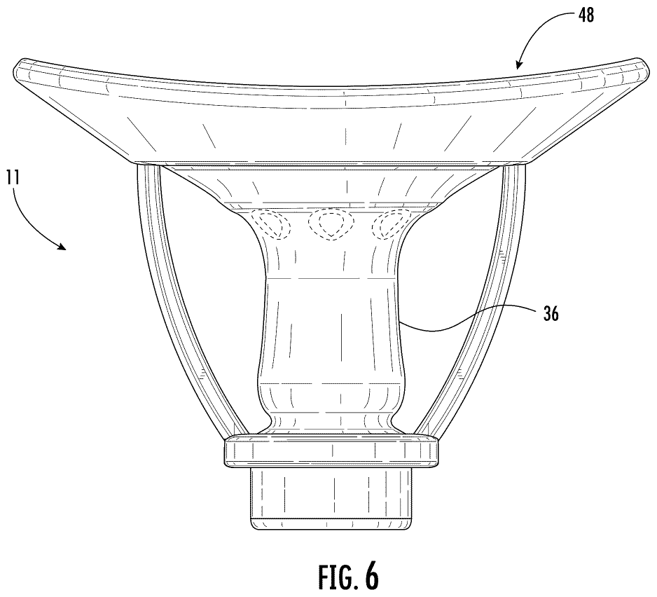

Alternative embodiments may include variations in the angle and curvature of the flexible outer ring 32 to accommodate different breast sizes and shapes. For example, in embodiments of the invention, and as shown in FIG. 6, a flange assembly 11 is shown having a flange interface 48 that is substantially ovular in shape. In embodiments, the flange takes the approximate shape of a Pringle chip. It will be understood that flexible tunnel 36 may be modified in terms of wall thickness, elasticity, or internal texture to optimize suction characteristics for different users.

The materials used in the construction of the breast pump flange 10 are selected to withstand repeated cleaning and sterilization methods, including boiling, steam sterilization, and dishwasher cleaning. All materials in contact with breast tissue or expressed milk comply with applicable food safety and medical device regulations.

While this invention has been described in conjunction with the embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A breast pump flange assembly comprising:
a structural skeleton comprising:
i) a base ring formed of a hard plastic material;
ii) a first post extending from the base ring;
iii) a second post extending from the base ring; and
iv) a flange ring connected to the first post and the second post, wherein the base ring, first post, second post, and flange ring are substantially rigid;
a flexible inner ring extending from an internal circumference of the flange ring;
d) a flexible tunnel comprising tunnel walls that define a lumen extending from the flexible inner ring to the base ring;
e) wherein, the tunnel walls are configured to at least partially contract when a pressure differential is created in the tunnel lumen.

2. The breast pump flange assembly of claim 1, wherein the first post and second post each have a first end attached to the base ring and a second end connected to the flange ring.

3. The breast pump flange assembly of claim 1, further comprising a flexible outer ring extending from an outer perimeter of the flange ring.

4. The breast pump flange assembly of claim 1, wherein the flexible tunnel has a wider diameter at its point of attachment to the flexible inner ring and tapers to a substantially cylindrical section that terminates at the base ring.

5. The breast pump flange assembly of claim 1, wherein the flexible tunnel walls are configured to move inward toward a longitudinal axis of the tunnel when negative pressure is applied.

6. The breast pump flange assembly of claim 1, wherein the structural skeleton provides rigidity while the flexible inner ring and the flexible tunnel provide dynamic response to pressure changes.

7. The breast pump flange assembly of claim 1, wherein the tunnel is configured to receive a nipple of a breast in at a first location within the tunnel lumen, and whereby the tunnel is configured to draw the nipple to a second location within the tunnel lumen responsive to reduced pressure in the tunnel lumen.

8. The breast pump assembly of claim 1, whereby the base ring and flexible inner ring form a flange interface.

9. The breast pump flange assembly of claim 8, wherein the flanger interface configured to receive a nipple of a breast in at a first location within the flange interface, and whereby the tunnel is configured to draw the nipple to a second location comprising an area within the tunnel lumen responsive to reduced pressure in the tunnel lumen.

10. The breast pump flange assembly of claim 1, wherein the tunnel walls are configured to draw nipple tissue further into the lumen when the pressure differential is applied.

11. The breast pump flange assembly of claim 8, wherein the flange interface is substantially oval shaped.
